(19) United States
(12) Patent Application Publication
(10) Pub. No.: US 2009/0306222 A1
(43) Pub. Date: Dec. 10, 2009

(54) FOOD SUPPLEMENTS AND USES THEREOF

(75) Inventors: Graham Burton, Ottawa (CA); Janusz Daroszewski, Ottawa (CA)

Correspondence Address: CLARK & ELBING LLP, 101 FEDERAL STREET, BOSTON, MA, 02110, US

(73) Assignee: Chemaphor Inc., Ontario (CA)

(21) Appl. No.: 12/225,995

(22) PCT Filed: Apr. 4, 2007
(86) PCT No.: PCT/CA2007/000563

§ 371 Date: Jun. 26, 2009

Related U.S. Application Data
(60) Provisional application No. 60/789,496, filed on Apr. 5, 2006.

(51) Int. Cl. A61K 31/015; C07C 13/28

(52) U.S. Cl. 514/763; 585/23

(57) ABSTRACT

The invention features compositions for administration of an oxidatively transformed carotenoid, fractionated oxidatively transformed carotenoid and components of oxidatively transformed carotenoid. The compositions are useful for supplementing the diet of an animal.

### BACKGROUND OF THE INVENTION

The invention relates to the use of carotenoid oxidation products to supplement the diet of an animal.

Animals raised under modern conditions optimized for growth promotion receive rations containing high proportions of protein, usually in the form of soybean or cottonseed meal, and high percentages of grains such as corn or milo, a type of sorghum. Feed additives which have been used include such hormones as diethylstilbestrol, or DES which also increases the rate of weight gain, and tranquilizers that prevent the disease or weight loss brought on by stressful confinement conditions. Routine antibiotic administration to animals has become almost universal since the discovery that the addition of small amounts of antibiotics such as penicillin, tetracycline and sulfamethazine, to animal feed increases the growth of pigs and cattle. Because feed is a relatively expensive cost factor in the production of food from animals (typically 50 to 70% of the cost), any improvement in the ability of the animal to convert feed into food products or enhancement in growth rate can directly improve the profitability of a food producer.

The use of such additives has not been without problems. One of the hormones that was commonly used as a growth stimulant, diethylstilbestrol, has been shown to be a carcinogen and has been banned from further use in most countries. Furthermore, the widespread use of antibiotics in animal feed promotes the development of antibiotic-resistant microorganisms.

As a result of the increasing appearance of antibiotic-resistant bacteria in feed lots and the potential for epidemics caused by antibiotic resistant bacteria, there is increasing governmental pressure to limit the use of antibiotics in animal feed. Consequently, there is an immediate and increasing need for new, safe, and effective growth stimulators of farm animals. There is a also a need for a method of improving the ability of animals to more efficiently convert their feed to body weight or other edible products and for new nutraceutical products for promoting general health and well being.

### SUMMARY OF THE INVENTION

The invention provides methods and compositions for supplementing the diet of an animal.

In a first aspect, the invention features a foodstuff including an additive selected from oxidatively transformed carotenoid and fractionated oxidatively transformed carotenoid. The additive can be fractionated oxidatively transformed carotenoid, such as a fraction including the polymeric component of oxidatively transformed carotenoid; or a fraction including a mixture of compounds, each of the compounds having a molecular weight of less than 700 Da, or less than 300 Da. Alternatively, the additive is oxidatively transformed carotenoid which has not been fractionated.

In a related aspect, the invention features a foodstuff including an additive selected from 1-(1,2,2-trimethylcyclopentyl)pent-2-ene-1,4-dione, 1-methylhydroxy-2,2,6-trimethylcyclohexene epoxide, 15,15′-epoxy-β-carotene, 2-(hydroxymethyl)-1,3,3-trimethylcyclohexane-1,2-diol, 2-(hydroxymethyl)-1,3,3-trimethylcyclohexanol, 2-hydroxy-2,6,6-trimethylcyclohexane-1-carboxaldehyde, 2-hydroxy-2,6,6-trimethylcyclohexanone, 2-methyl-6-oxo-2,4-heptadienal, 2-methylhept-2-en-4-one, 2,2-dimethyl-6-methylenecyclohexanone, 2,2,6-cyclohexenyl-1-formate, 2,2,6-cyclohexenyl-1-formate epoxide, 2,2,6-trimethylcyclohexene, 2,2,6-trimethylcyclohexene epoxide, 2,5,5,8a-tetramethyl-6,7,8,8a-tetrahydro-2H-chromen-3(5H)-one, 2,6,6-trimethylcyclohex-2-enone, 2,6,6,-trimethylcyclohexanone, 3-hydroxy-1-(2,6,6-trimethylcyclohex-1-enyl)butan-2-one, 4-ethylbenzaldehyde, 4-oxo-β-apo-13-carotenone, 4-oxo-β-ionone, 4-oxo-β-ionylideneacetaldehyde, 5,6-dihydroxy-5,6-dihydro-β-ionone, 5,6-epoxy-β-carotene, 5,6,5′,6′-diepoxy-β-carotene, 5,6,5′,8′-diepoxy-β-carotene, 5,8-epoxy-β-carotene, 5,8,5′,8′-diepoxy-β-carotene, 6-hydroxy-alpha-ionone, 6-hydroxy-gamma-ionone, 6-methyl-6-(5-methylfuran-2-yl)heptan-2-one, 6-methylhept-5-en-2-one, 6-methylhept-6-en-2-one, 6-methylhepta-3,5-dien-2-one, 6-methylheptan-2-one, 6,6-dimethylundec-3-ene-2,5,10-trione, alpha-ionone, cetoisophorone, dihydroactinidiolide, geranial, neral, pseudo-ionone, retinal, retinal 5,6-epoxide, retro-gamma-ionone, semi-β-carotenone, Sapo-10′-carotenal, 6-apo-12′-carotenal, β-apo-13-carotenone, β-apo-13-carotenone 5,6-epoxide, β-apo-14-carotenol, β-apo-1,4′-carotenal, β-apo-8′-carotenal, β-carotenone, β-cyclocitral, β-cyclocitral epoxide, β-damascone, β-ionone, β-ionone 5,6-epoxide, β-ionylideneacetaldehyde, β-ionylideneacetaldehyde 5,6-epoxide, β-methylionone, and mixtures thereof, wherein the foodstuff includes from 0.0000001% and 0.00001% (w/w) of the additive. Desirably, the foodstuff includes from 0.0000001% to 0.001% (w/w) of the compound. Desirably, the foodstuff contains from 0.0000001% to 0.0005%, 0.0000001% to 0.0001%, 0.0000001% to 0.00005%, 0.0000001% to 0.00001%, 0.0000001% to 0.000005%, or 0.0000001% (1 ppb) to 0.000001% (10 ppb)(w/w) of the additive.

In still another aspect, the invention features a method of supplementing the diet of an animal by feeding said animal a food additive selected from oxidatively transformed carotenoid and fractionated oxidatively transformed carotenoid. The additive can be fractionated oxidatively transformed carotenoid, such as a fraction including the polymeric component of oxidatively transformed carotenoid; or a fraction including a mixture of compounds, each of the compounds having a molecular weight of less than 700 Da, or less than 300 Da. Alternatively, the additive is oxidatively transformed carotenoid which has not been fractionated. In one embodiment, the additive is admixed with a foodstuff and fed to the animal.

The invention also features a kit, including: (i) a composition including a food additive selected from oxidatively transformed carotenoid and fractionated oxidatively transformed carotenoid; and (ii) instructions for administering the additive to an animal. The additive can be fractionated oxidatively transformed carotenoid, such as a fraction including the polymeric component of oxidatively transformed carotenoid; or a fraction including a mixture of compounds, each of the compounds having a molecular weight of less than 700 Da, or less than 300 Da. Alternatively, the additive is oxidatively transformed carotenoid which has not been fractionated. In one embodiment, the composition in part (i) of the kit includes a bulking agent and wherein from 0.5% to 50% (w/w) of the composition is the food additive. Desirably, the composition includes from 1% to 50%, 1% to 40%, 5% to 40%, 10% to 40%, or 15% to 30% (w/w) of the food additive. In another embodiment, the kit further includes instruction for mixing the composition with an animal feed.

The invention further features a food supplement including: (a) a vitamin selected from vitamin C, vitamin D, vitamin E, vitamin K, folate, vitamin B6, and vitamin B12; and (b) oxidatively transformed carotenoid or a component thereof. In certain embodiments, the food supplement is formulated in a unit dosage form containing from about 5% to 1000%, 5% to 5000%, 50% to 5000%, 50% to 1000%, 100% to 5000%, 200% to 5000%, 5% to 500%, 5% to 100%, 50% to 10000%, 100% to 10000%, or even 500% to 10000% of the RDA of the vitamin and from about 10 μg to 100 mg, 100 μg to 100 mg, 100 μg to 50 mg, 100 μg to 25 mg, 10 μg to 50 mg, 10 μg to 5 mg, 200 μg to 10 mg, 200 μg to 250 mg, 200 μg to 250 mg, or even 100 μg to 250 mg of the oxidatively transformed carotenoid or a component thereof.

The invention also features a food supplement including: (a) a mineral selected from calcium, chromium, copper, fluoride, iodine, iron, magnesium, manganese, molybdenum, phosphorus, potassium, selenium, sodium, and zinc; and (b) oxidatively transformed carotenoid or a component thereof. In certain embodiments, the food supplement is formulated in a unit dosage form containing from about 5% to 500%, 5% to 5000%, 50% to 5000%, 50% to 1000%, 100% to 5000%, 200% to 5000%, 5% to 250%, 5% to 100%, 50% to 10000%, 100% to 10000%, or even 500% to 10000% of the RDA of the mineral and from about 10 μg to 100 mg, 100 μg to 100 mg, 100 μg to 50 mg, 100 μg to 25 mg, 10 μg to 50 mg, 10 μg to 5 mg, 200 μg to 10 mg, 200 μg to 250 mg, 200 μg to 250 mg, or even 100 μg to 250 mg of the oxidatively transformed carotenoid or a component thereof.

In a related aspect, the invention further features a food supplement including: (a) an omega fatty acid selected from alpha-linolenic acid, stearidonic acid, eicosatetraenoic acid, eicosapentaenoic acid, docosahexaenoic acid, linoleic acid, gamma-linolenic acid, dihomo-gamma-linolenic acid, and arachidonic acid; and (b) oxidatively transformed carotenoid or a component thereof. In certain embodiments, the food supplement is formulated in a unit dosage form containing from about 10 mg to 2 g, 1 mg to 2 g, 10 mg to 1 g, 1 mg to 1 g, 10 mg to 500 mg, 1 mg to 500 mg, 100 mg to 1 g, or even 100 mg to 500 of the omega fatty acid and from about 10 μg to 100 mg, 100 μg to 100 mg, 100 μg to 50 mg, 100 μg to 25 mg, 10 μg to 50 mg, 10 μg to 5 mg, 200 μg to 10 mg, 200 μg to 250 mg, 200 μg to 250 mg, or even 100 μg to 250 mg of the oxidatively transformed carotenoid or a component thereof.

The invention also features a food supplement including: (a) an amino acid selected from isoleucine, leucine, lysine, methionine, phenylalanine, threonine, tryptophan, and valine; and (b) oxidatively transformed carotenoid or a component thereof. In certain embodiments, the food supplement is formulated in a unit dosage form containing from about 5% to 500%, 5% to 5000%, 50% to 5000%, 50% to 1000%, 100% to 5000%, 200% to 5000%, 5% to 250%, 5% to 100%, 50% to 10000%, 100% to 10000%, or even 500% to 10000% of the RDA of the amino acid and from about 10 μg to 100 mg, 100 μg to 100 mg, 100 μg to 50 mg, 100 μg to 25 mg, 10 μg to 50 mg, 10 μg to 5 mg, 200 μg to 10 mg, 200 μg to 250 mg, 200 μg to 250 mg, or even 100 μg to 250 mg of the oxidatively transformed carotenoid or a component thereof.

In still another related aspect, the invention features a food supplement including: (a) an herb selected from angelica, astragalus, avena sativa, bayberry bark, billberry, black cohosh, black haw, black walnut, blessed thistle, blue cohosh, blue vervain, buchu, buckthorn, burdock, cascara sagada, casteberry, cayenne, chamomille, chaparral, chaste tree, chickweed, cloves, coltsfoot, comphrey root, cornsilk, cough calm, crampbark, damiana, dandelion, dandelion root, dill seed, dong quai, echinacea, elecampane, essiac, eucalyptus, fennel, fenugreek, gentian, ginger, ginkgo, ginseng, goldenseal, gota kola, guarana, hawthorne berry, hops, horehound, horsetail, hydrangea, hyssop, kelp, kola nut, licorice, lobelia, maca, marshmallow, motherwort, muira puama, mullien, myrrh, nettle, oatstraw, oregon grape root, parsley, passion flower, pau d'arco, peppermint, plantain, pleurisy root, prickley ash bark, red clover, red raspberry, sarsaparilla, saw palmetto, schizandra, scullcap, sheep sorrel, slippery elm, squawvine, St. Johns wort, tumeric, turkey rhubarb, valerian, white willow bark, wild cherry bark, wild yam, yarrow, yellow dock, yohimbi, and extracts thereof; and (b) oxidatively transformed carotenoid or a component thereof. In certain embodiments, the food supplement is formulated in a unit dosage form containing from about 1 mg to 250 mg, 1 mg to 1 g, 1 mg to 2 g, 1 mg to 100 mg, 1 mg to 500 mg, 10 mg to 100 mg, 10 mg to 250 mg, 50 mg to 250 mg, 50 mg to 500 mg, 50 mg to 1 g, 100 mg to 500 mg, or even 100 mg to 750 mg of the herb and from about 10 μg to 100 mg, 100 μg to 100 mg, 100 μg to 50 mg, 100 μg to 25 mg, 10 μg to 50 mg, 10 μg to 5 mg, 200 μg to 10 mg, 200 μg to 250 mg, 200 μg to 250 mg, or even 100 μg to 250 mg of the oxidatively transformed carotenoid or a component thereof.

The invention further features a food supplement formulated in unit dosage form containing from 100 μg to 100 mg oxidatively transformed carotenoid or a component thereof. In certain embodiments, the food supplement in a unit dosage form contains from about 10 μg to 100 mg, 100 μg to 100 mg, 100 μg to 50 mg, 100 μg to 25 mg, 10 μg to 50 mg, 10 μg to 5 mg, 200 μg to 10 mg, 200 μg to 250 mg, 200 μg to 250 mg, or even 100 μg to 250 mg of the oxidatively transformed carotenoid or a component thereof. For example, each dosage can contain 100 μg, 200 μg, 300 μg, 400 μg, 500 μg, 600 μg, 700 μg, 800 μg, 900 μg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 60 mg, 70 mg, 80 mg, 90 mg, or even 100 mg of the oxidatively transformed carotenoid or a component thereof.

In certain embodiments of the food supplements of the invention, the food supplement is formulated in unit dosage form as a tablet, pill, capsule, or caplet. In still other embodiments, the food supplement is formulated as a liquid or a powder containing from 0.00001% and 0.005% (w/w) of the oxidatively transformed carotenoid or a component thereof. Desirably, the food supplement contains between 0.00001% and 0.05%, 0.00001% and 0.01%, 0.00001% and 0.005%, 0.00001% and 0.001%, 0.00001% and 0.0005%, or 0.00001% and 0.0001% (w/w) oxidatively transformed carotenoid, or a component thereof, or fractionated oxidatively transformed carotenoid. In yet another embodiment, the food supplement includes the polymeric component of oxidatively transformed carotenoid. In certain embodiments of the food supplements of the invention, the oxidatively transformed carotenoid or a component thereof is selected from 1-(1,2,2-trimethylcyclopentyl)pent-2-ene-1,4-dione, 1-methylhydroxy-2,2,6-trimethylcyclohexene epoxide, 15,15′-epoxy-β-carotene, 2-(hydroxymethyl)-1,3,3-trimethylcyclohexane-1,2-diol, 2-(hydroxymethyl)-1,3,3-trimethylcyclohexanol, 2-hydroxy-2,6,6-trimethylcyclohexane-1-carboxaldehyde, 2-hydroxy-2,6,6-trimethylcyclohexanone, 2-methyl-6-oxo-2,4-heptadienal, 2-methylhept-2-en-4-one, 2,2-dimethyl-6-methylenecyclohexanone, 2,2,6-cyclohexenyl-1-formate, 2,2,6-cyclohexenyl-1-formate epoxide, 2,2,6-trimethylcyclohexene, 2,2,6-trimethylcyclohexene epoxide, 2,5,5,8a-tetramethyl-6,7,8,8a-tetrahydro-2H-chromen-3(5H)-one, 2,6,6-trimethylcyclohex-2-enone, 2,6,6,-trimethylcyclohexanone, 3-hydroxy-1-(2,6,6-trimethylcyclohex-1-enyl)butan-2-one, 4-ethylbenzaldehyde, 4-oxo-β-apo-13-carotenone, 4-oxo-β-ionone, 4-oxo-β-ionylideneacetaldehyde, 5,6-dihydroxy-5,6-dihydro-β-ionone, 5,6-epoxy-β-carotene, 5,6,5′,6′-diepoxy-β-carotene, 5,6,5′,8′-diepoxy-1β-carotene, 5,8-epoxy-β-carotene, 5,8,5′,8′-diepoxy-β-carotene, 6-hydroxy-alpha-ionone, 6-hydroxy-gamma-ionone, 6-methyl-6-(5-methylfuran-2-yl)heptan-2-one, 6-methylhept-5-en-2-one, 6-methylhept-6-en-2-one, 6-methylhepta-3,5-dien-2-one, 6-methylheptan-2-one, 6,6-dimethylundec-3-ene-2,5,10-trione, alpha-ionone, cetoisophorone, dihydroactinidiolide, geranial, neral, pseudo-ionone, retinal, retinal 5,6-epoxide, retro-gamma-ionone, semi-β-carotenone, β-apo-10′-carotenal, β-apo-12′-carotenal, β-apo-13-carotenone, 1-apo-13-carotenone 5,6-epoxide, β-apo-14-carotenol, β-apo-1,4′-carotenal, β-apo-8′-carotenal, β-carotenone, 1-cyclocitral, 13-cyclocitral epoxide, β-damascone, β-ionone, β-ionone 5,6-epoxide, β-ionylideneacetaldehyde, β-ionylideneacetaldehyde 5,6-epoxide, β-methylionone, and mixtures thereof. Desirably, the oxidatively transformed carotenoid or a component thereof is 2-methyl-6-oxo-2,4-heptadienal.

The invention also features a kit, including: (i) a food supplement of the invention; and (ii) instructions for administering the food supplement to an animal.

The invention further features a method of supplementing the diet of an animal by administering to the animal a food supplement of the invention.

In an embodiment of any of the above aspects, the fractionated oxidatively transformed carotenoid includes a compound selected from 1-(1,2,2-trimethylcyclopentyl)pent-2-ene-1,4-dione, 1-methylhydroxy-2,2,6-trimethylcyclohexene epoxide, 15,15′-epoxy-β-carotene, 2-(hydroxymethyl)-1,3,3-trimethylcyclohexane-1,2-diol, 2-(hydroxymethyl)-1,3,3-trimethylcyclohexanol, 2-hydroxy-2,6,6-trimethylcyclohexane-1-carboxaldehyde, 2-hydroxy-2,6,6-trimethylcyclohexanone, 2-methyl-6-oxo-2,4-heptadienal, 2-methylhept-2-en-4-one, 2,2-dimethyl-6-methylenecyclohexanone, 2,2,6-cyclohexenyl-1-formate, 2,2,6-cyclohexenyl-1-formate epoxide, 2,2,6-trimethylcyclohexene, 2,2,6-trimethylcyclohexene epoxide, 2,5,5,8a-tetramethyl-6,7,8,8a-tetrahydro-2H-chromen-3(5H)-one, 2,6,6-trimethylcyclohex-2-enone, 2,6,6,-trimethylcyclohexanone, 3-hydroxy-1-(2,6,6-trimethylcyclohex-1-enyl)butan-2-one, 4-ethylbenzaldehyde, 4-oxo-β-apo-13-carotenone, 4-oxo-β-ionone, 4-oxo-β-ionylideneacetaldehyde, 5,6-dihydroxy-5,6-dihydro-β-ionone, 5,6-epoxy-β-carotene, 5,6,5′,6′-diepoxy-β-carotene, 5,6,5′,8′-diepoxy-β-carotene, 5,8-epoxy-β-carotene, 5,8,5′,8′-diepoxy-β-carotene, 6-hydroxy-alpha-ionone, 6-hydroxy-gamma-ionone, 6-methyl-6-(5-methylfuran-2-yl)heptan-2-one, 6-methylhept-5-en-2-one, 6-methylhept-6-en-2-one, 6-methylhepta-3,5-dien-2-one, 6-methylheptan-2-one, 6,6-dimethylundec-3-ene-2,5,10-trione, alpha-ionone, cetoisophorone, dihydroactinidiolide, geranial, neral, pseudo-ionone, retinal, retinal 5,6-epoxide, retro-gamma-ionone, semi-β-carotenone, β-apo-10′-carotenal, β-apo-12′-carotenal, β-apo-13-carotenone, β-apo-13-carotenone 5,6-epoxide, 1-apo-14-carotenol, β-apo-1,4′-carotenal, β-apo-8′-carotenal, β-carotenone, β-cyclocitral, 13-cyclocitral epoxide, β-damascone, β-ionone, β-ionone 5,6-epoxide, β-ionylideneacetaldehyde, β-ionylideneacetaldehyde 5,6-epoxide, and 3-methylionone. The foodstuff can include from 0.0000001% to 0.001% (w/w) of the compound. Desirably, the foodstuff contains from 0.0000001% to 0.0005%, 0.0000001% to 0.0001%, 0.0000001% to 0.00005%, 0.0000001% to 0.00001%, 0.0000001% to 0.000005%, or 0.0000001% (1 ppb) to 0.000001% (10 ppb) (w/w) of the compound.

In any of the above aspects, the foodstuff can include from 0.00001% to 0.1% (w/w) oxidatively transformed carotenoid, or a component thereof, or fractionated oxidatively transformed carotenoid. Desirably, the foodstuff contains between 0.00001% and 0.05%, 0.00001% and 0.01%, 0.00001% and 0.005%, 0.00001% and 0.001%, 0.00001% and 0.0005%, or 0.00001% and 0.0001% (w/w) oxidatively transformed carotenoid, or a component thereof, or fractionated oxidatively transformed carotenoid.

The invention also features a method of promoting weight gain in an animal by administering to the animal oxidatively transformed carotenoid, a component thereof, or fractionated oxidatively transformed carotenoid in an amount effective to promote weight gain.

The invention further features a method of increasing feed conversion efficiency in an animal by administering to the animal oxidatively transformed carotenoid, a component thereof, or fractionated oxidatively transformed carotenoid in an amount effective to increase feed conversion efficiency.

In an embodiment of any of the aspects described herein, the oxidatively transformed carotenoid is used without fractionation of the mixture. Alternatively, a composition including the polymeric component of oxidatively transformed carotenoid or a composition including 1-(1,2,2-trimethylcyclopentyl)pent-2-ene-1,4-dione, 1-methylhydroxy-2,2,6-trimethylcyclohexene epoxide, 15,15′-epoxy-β-carotene, 2-(hydroxymethyl)-1,3,3-trimethylcyclohexane-1,2-diol, 2-(hydroxymethyl)-1,3,3-trimethylcyclohexanol, 2-hydroxy-2,6,6-trimethylcyclohexane-1-carboxaldehyde, 2-hydroxy-2,6,6-trimethylcyclohexanone, 2-methyl-6-oxo-2,4-heptadienal, 2-methylhept-2-en-4-one, 2,2-dimethyl-6-methylenecyclohexanone, 2,2,6-cyclohexenyl-1-formate, 2,2,6-cyclohexenyl-1-formate epoxide, 2,2,6-trimethylcyclohexene, 2,2,6-trimethylcyclohexene epoxide, 2,5,5,8a-tetramethyl-6,7,8,8a-tetrahydro-2H-chromen-3(5H)-one, 2,6,6-trimethylcyclohex-2-enone, 2,6,6,-trimethylcyclohexanone, 3-hydroxy-1-(2,6,6-trimethylcyclohex-1-enyl)butan-2-one, 4-ethylbenzaldehyde, 4-oxo-β-apo-13-carotenone, 4-oxo-β-ionone, 4-oxo-β-ionylideneacetaldehyde, 5,6-dihydroxy-5,6-dihydro-β-ionone, 5,6-epoxy-β-carotene, 5,6,5′,6′-diepoxy-β-carotene, 5,6,5′,8′-diepoxy-β-carotene, 5,8-epoxy-β-carotene, 5,8,5′,8′-diepoxy-β-carotene, 6-hydroxy-alpha-ionone, 6-hydroxy-gamma-ionone, 6-methyl-6-(5-methylfuran-2-yl)heptan-2-one, 6-methylhept-5-en-2-one, 6-methylhept-6-en-2-one, 6-methylhepta-3,5-dien-2-one, 6-methylheptan-2-one, 6,6-dimethylundec-3-ene-2,5,10-trione, alpha-ionone, cetoisophorone, dihydroactinidiolide, geranial, neral, pseudo-ionone, retinal, retinal 5,6-epoxide, retro-gamma-ionone, semi-β-carotenone, β-apo-10′-carotenal, β-apo-12′-carotenal, β-apo-13-carotenone, β-apo-13-carotenone 5,6-epoxide, 8-apo-14-carotenol, β-apo-14′-carotenal, β-apo-8′-carotenal, β-carotenone, β-cyclocitral, β-cyclocitral epoxide, β-damascone, β-ionone, β-ionone 5,6-epoxide, β-ionylideneacetaldehyde, β-ionylideneacetaldehyde 5,6-epoxide, β-methylionone, or mixtures thereof can be used in the methods, kits, and foodstuffs of the invention. Desirably, the component of oxidatively transformed carotenoid used includes the polymeric component and/or 2-methyl-6-oxo-2,4-heptadienal.

In another embodiment of any of the aspects described herein, the animal is selected from humans, dogs, cats, horses, sheep, swine, cattle, poultry, and fish.

In an embodiment of any of the above methods, oxidatively transformed carotenoid, component thereof, or fractionated oxidatively transformed carotenoid is administered orally, by injection, or by aerosol. Desirably, the oxidatively transformed carotenoid, component thereof, or fractionated oxidatively transformed carotenoid is admixed with a foodstuff and fed to the animal.

Foodstuffs of the invention include, without limitation, baked goods, beverages, beverage mixes, health bars, biscuits, and animal feeds. The animal feed may be a dry or semi-moist pet food, or feed for an agricultural animal, such as horse feed, swine feed (e.g., nursery/starter swine feed, grow-finish swine feed, or breeding herd swine feed), poultry feed (e.g., turkey poultry feed, broilers poultry feed, or breeders poultry feed), sheep feed, cattle feed (e.g., dairy cattle feed or beef cattle feed), or fish feed (e.g., tilapia feed, catfish feed, trout feed, or salmon feed).

Foodstuffs of the invention may further include an antioxidant. Exemplary antioxidants include, without limitation, beta-carotene, vitamin E, vitamin C, butylated hydroxytoluene, butylated hydroxyanisole, tertiary-butylhydroquinone, propyl gallate, and ethoxyquin.

In another embodiment of any of the above aspects, the foodstuffs of the invention further include a medicament, such as an antibiotic or hormone. Such medicaments can be added in amounts typically found in commercial feeds.

As used herein, an “amount effective to promote weight gain” is an amount of oxidatively transformed carotenoid, a component thereof, or fractionated oxidatively transformed carotenoid which causes an animal to gain weight faster in comparison to an animal of the same species and age which is raised under the same conditions and receives the same diet without oxidatively transformed carotenoid, a component thereof, or fractionated oxidatively transformed carotenoid. The average increase in mass is greater than 0.5%, preferably greater than 1%, 2%, 3%, 4%, or even 5% in comparison to the control animal.

As used herein, an “amount effective to increase feed conversion efficiency” is an amount of oxidatively transformed carotenoid, a component thereof, or fractionated oxidatively transformed carotenoid which causes an increase in feed conversion efficiency in comparison to an animal of the same species and age which is raised under the same conditions and receives the same diet without oxidatively transformed carotenoid, a component thereof, or fractionated oxidatively transformed carotenoid. The average reduction in feed needed to produce the same weight is greater than 0.5%, preferably greater than 1%, 2%, 3%, 4%, or even 5% in comparison to the control animal.

By “animal” is meant any animal including, without limitation, humans, dogs, cats, horses, sheep, swine, cattle, poultry, and fish.

As used herein, “carotenoid” refers to naturally-occurring pigments of the terpenoid group that can be found in plants, algae, bacteria, and certain animals, such as birds and shellfish. Carotenoids include carotenes, which are hydrocarbons (i.e., without oxygen), and their oxygenated derivatives (i.e., xanthophylls). Examples of carotenoids include lycopene; beta-carotene; zeaxanthin; echinenone; isozeaxanthin; astaxanthin; canthaxanthin; lutein; citranaxanthin; β-apo-8′-carotenic acid ethyl ester; hydroxy carotenoids, such as alloxanthin, apocarotenol, astacene, astaxanthin, capsanthin, capsorubin, carotenediols, carotenetriols, carotenols, cryptoxanthin, decaprenoxanthin, epilutein, fucoxanthin, hydroxycarotenones, hydroxyechinenones, hydroxylycopene, lutein, lycoxanthin, neurosporine, phytoene, phytofluoene, rhodopin, spheroidene, torulene, violaxanthin, and zeaxanthin; and carboxylic carotenoids, such as apocarotenoic acid, β-apo-8′-carotenoic acid, azafrin, bixin, carboxylcarotenes, crocetin, diapocarotenoic acid, neurosporaxanthin, norbixin, and lycopenoic acid.

As used herein, the term “oxidatively transformed carotenoid” refers to a carotenoid which has been reacted with up to 6 to 8 molar equivalents of oxygen, or an equivalent amount of oxygen from another oxidizing agent, resulting in a mixture of very low molecular weight oxidative cleavage products and a large proportion of polymeric material (i.e., that component of the oxidatively transformed carotenoid having a molecular weight of greater than 700 Daltons). The resulting reaction produces a mixture that includes molecular species having molecular weights ranging from about 100 to 8,000 Daltons. The polymeric material is believed to be formed by the many possible chemical recombinations of the various oxidative fragments that are formed. Methods of making oxidatively transformed carotenoid are described in U.S. Pat. No. 5,475,006 and U.S. Ser. No. 08/527,039, each of which are incorporated herein by reference.

As used herein “component” refers to an active oxidized component of an oxidatively transformed carotenoid mixture that includes either polymeric material or a compound selected from 1-(1,2,2-trimethylcyclopentyl)pent-2-ene-1,4-dione, 1-methylhydroxy-2,2,6-trimethylcyclohexene epoxide, 15,15′-epoxy-β-carotene, 2-(hydroxymethyl)-1,3,3-trimethylcyclohexane-1,2-diol, 2-(hydroxymethyl)-1,3,3-trimethylcyclohexanol, 2-hydroxy-2,6,6-trimethylcyclohexane-1-carboxaldehyde, 2-hydroxy-2,6,6-trimethylcyclohexanone, 2-methyl-6-oxo-2,4-heptadienal, 2-methylhept-2-en-4-one, 2,2-dimethyl-6-methylenecyclohexanone, 2,2,6-cyclohexenyl-1-formate, 2,2,6-cyclohexenyl-1-formate epoxide, 2,2,6-trimethylcyclohexene, 2,2,6-trimethylcyclohexene epoxide, 2,5,5,8a-tetramethyl-6,7,8,8a-tetrahydro-2H-chromen-3(5H)-one, 2,6,6-trimethylcyclohex-2-enone, 2,6,6,-trimethylcyclohexanone, 3-hydroxy-1-(2,6,6-trimethylcyclohex-1-enyl)butan-2-one, 4-ethylbenzaldehyde, 4-oxo-β-apo-13-carotenone, 4-oxo-β-ionone, 4-oxo-β-ionylideneacetaldehyde, 5,6-dihydroxy-5,6-dihydro-β-ionone, 5,6-epoxy-β-carotene, 5,6,5′,6′-diepoxy-β-carotene, 5,6,5′,8′-diepoxy-β-carotene, 5,8-epoxy-β-carotene, 5,8,5′,8′-diepoxy-β-carotene, 6-hydroxy-alpha-ionone, 6-hydroxy-gamma-ionone, 6-methyl-6-(5-methylfuran-2-yl)heptan-2-one, 6-methylhept-5-en-2-one, 6-methylhept-6-en-2-one, 6-methylhepta-3,5-dien-2-one, 6-methylheptan-2-one, 6,6-dimethylundec-3-ene-2,5,10-trione, alpha-ionone, cetoisophorone, dihydroactinidiolide, geranial, neral, pseudo-ionone, retinal, retinal 5,6-epoxide, retro-gamma-ionone, semi-β-carotenone, β-apo-10′-carotenal, β-apo-12′-carotenal, β-apo-13-carotenone, β-apo-13-carotenone 5,6-epoxide, β-apo-14-carotenol, β-apo-14′-carotenal, β-apo-8′-carotenal, β-carotenone, β-cyclocitral, β-cyclocitral epoxide, β-damascone, β-ionone, β-ionone 5,6-epoxide, β-ionylideneacetaldehyde, β-ionylideneacetaldehyde 5,6-epoxide, β-methylionone, and mixtures thereof. Components of oxidatively transformed carotenoid can be capable of either increasing feed conversion efficiency in an animal or promoting weight gain in an animal, or both. Methods for assessing whether a particular fraction of oxidatively transformed carotenoid is capable of increasing feed conversion efficiency or promoting weight gain are provided in the Examples. Methods of fractionating oxidatively transformed carotenoid mixtures into components (e.g., fractions containing polymeric component, fractions in which the compounds in the mixture each have a MW of less than 700 Da, or 300 Da) are described in U.S. Pat. No. 5,475,006 and U.S. Ser. No. 08/527,039, each of which are incorporated herein by reference.

As used herein, the term “recommended daily allowance” or “RDA” refers to the amount of a nutrient recited in the table below.

| Nutrient | RDA | |
|---|---|---|
| Vitamin A | 900 | μg/day |
| Vitamin C | 90 | mg/day |
| Vitamin D | 5 | μg/day |
| Vitamin E | 15 | mg/day |
| Vitamin K | 120 | μg/day |
| Folate | 400 | μg/day |
| Vitamin B6 | 1.3 | mg/day |
| Vitamin B12 | 2.4 | μg/day |
| Calcium | 1000 | mg/day |
| Chloride | 2300 | mg/day |
| Chromium | 35 | μg/day |
| Copper | 900 | μg/day |
| Fluoride | 4 | mg/day |
| Iodine | 150 | μg/day |
| Iron | 8 | mg/day |
| Magnesium | 400 | mg/day |
| Manganese | 2.3 | mg/day |
| Molybdenum | 45 | μg/day |
| Phosphorus | 700 | mg/day |
| Potassium | 4700 | mg/day |
| Selenium | 55 | μg/day |
| Sodium | 1500 | mg/day |
| Zinc | 11 | mg/day |
| Phenylalanine | 980 | mg/day |
| Leucine | 980 | mg/day |
| Methionine | 910 | mg/day |
| Lysine | 840 | mg/day |
| Isoleucine | 700 | mg/day |
| Valine | 700 | mg/day |
| Threonine | 490 | mg/day |
| Tryptophan | 245 | mg/day |

The term “unit dosage form” refers to physically discrete units suitable as unitary dosages for a subject, each unit containing a predetermined quantity of oxidatively transformed carotenoid or a component thereof, typically in amounts of 100 μg to 100 mg, in association with a pharmaceutically acceptable excipient.

The synthesis and purification of 2-methyl-6-oxo-2,4-heptadienal has been reported in U.S. Ser. No. 08/527,039. A more convenient five-step synthetic scheme for the preparation of 2-methyl-6-oxo-2,4-heptadienal is provided in U.S. Ser. No. 10/196,695, published May 22, 2003.

The compositions and methods of the invention can be used to promote weight gain and increase feed conversion efficiency in animals.

Other features and advantages of the invention will be apparent from the following Detailed Description and the claims.

### DETAILED DESCRIPTION

The invention provides foodstuffs and food supplements for the administration of oxidatively transformed carotenoid or fractionated oxidatively transformed carotenoid. The foodstuffs can be useful for supplementing the diet of an animal and useful as a nutraceutical for promoting general health and well being.

### Administration

The oxidatively transformed carotenoid, a component thereof, or fractionated oxidatively transformed carotenoid can be administered in an amount effective to promote weight gain or effective to increase feed conversion efficiency. For oxidatively transformed carotenoid, typical dose ranges are from about 1 μg/kg to about 100 mg/kg of body weight per day. Desirably, a dose of between 5 μg/kg and 50 mg/kg of body weight, or 5 μg/kg and 5 mg/kg of body weight, is administered daily. For a component of oxidatively transformed carotenoid, typical dose ranges are from about 0.05 μg/kg to about 500 μg/kg of body weight per day. Desirably, a dose of between 0.05 μg/kg and 50 μg/kg of body weight, or 0.05 μg/kg and 5 μg/kg of body weight, is administered daily. The dosage of oxidatively transformed carotenoid, a component thereof, or fractionated oxidatively transformed carotenoid to be administered is likely to depend on such variables as the species, diet, and age of the animal. Standard trials, such as those described in Example 1 may be used to optimize the dose and dosing frequency of the oxidatively transformed carotenoid or fractionated oxidatively transformed carotenoid.

Oxidatively transformed carotenoid, a component thereof, or fractionated oxidatively transformed carotenoid may be administered orally, by injection, or by aerosol. When injected, the administration can be parenteral, intravenous, intra-arterial, subcutaneous, intramuscular, intracranial, intraorbital, intraventricular, intracapsular, intraspinal, intracisternal, or intraperitoneal.

Oxidatively transformed carotenoid, a component thereof, or fractionated oxidatively transformed carotenoid may be added to a foodstuff or formulated with a pharmaceutically acceptable diluent, carrier, or excipient as described in U.S. Ser. No. 10/196,695, published May 22, 2003. Pharmaceutical formulations may, for example, be in the form of liquid solutions or suspensions; for oral administration, formulations may be in the form of tablets or capsules; and for intranasal formulations, in the form of powders, nasal drops, or aerosols. Methods well known in the art for making formulations are found, for example, in “Remington: The Science and Practice of Pharmacy” (20th ed., ed. A. R. Gennaro, 2000, Lippincott Williams & Wilkins).

In certain embodiments, the food supplements of the invention can be formulated using microencapsulation techniques as described in, for example, Schrooyen at al., Proc. Nutr. Soc. 60:475 (2001).

Desirably, oxidatively transformed carotenoid, a component thereof, or fractionated oxidatively transformed carotenoid is admixed with a foodstuff and fed to the animal.

### Foodstuffs

Oxidatively transformed carotenoid, a component thereof, or fractionated oxidatively transformed carotenoid can be admixed with a foodstuff and fed to the animal in an amount effective to promote weight gain or effective to increase feed conversion efficiency.

In preparing a foodstuff of the invention, the oxidatively transformed carotenoid, a component thereof, or fractionated oxidatively transformed carotenoid is optionally admixed with a bulking agent prior to being added to the foodstuff. Bulking agents include, without limitation, starch, protein, fats, and mixtures thereof. Desirably, the bulking agent is selected from corn starch, whey, flour, sugar, soybean meal, maltodextrin, and guar gum.

Foodstuffs of the invention can also include antioxidants to prevent further oxidation of the oxidatively transformed carotenoid or a component thereof. Oxidation can be prevented by the introduction of naturally-occurring antioxidants, such as beta-carotene, vitamin E, vitamin C, and tocopherol or of synthetic antioxidants such as butylated hydroxytoluene, butylated hydroxyanisole, tertiary-butylhydroquinone, propyl gallate or ethoxyquin to the foodstuff. The amount of antioxidants incorporated in this manner depends on requirements such as product formulation, shipping conditions, packaging methods, and desired shelf-life.

Animal Feeds

Animal feeds of the present invention can contain oxidatively transformed carotenoid, or a component thereof, or fractionated oxidatively transformed carotenoid. The animal feeds are generally formulated to provide nutrients in accordance with industry standards. The feeds may be formulated from a variety of different feed ingredients, which are chosen according to market price and availability. Accordingly, some components of the feed may change over time. For discussions on animal feed formulations and NRC guidelines, see Church, Livestock Feeds and Feeding, O&B Books, Inc., Corvallis Oreg. (1984) and Feeds and Nutrition Digest, Ensminger, Oldfield and Heineman eds., Ensminger Publishing Corporation, Clovis, Calif. (1990), each of which is incorporated herein by reference.

Swine and other animal feeds are traditionally balanced based upon protein and energy requirements, and then adjusted if needed to meet the other requirements, which will vary for the different stages of growth and maintenance of the animal. Growing young animals will require higher protein feeds, while finishing animals close to market will require higher energy, high carbohydrate, feeds. For example, typical hog prestarter, starter and grower-finisher feeds will generally contain about 20-24% protein, 18-20% protein and 13-17% protein respectively. In some feeding situations, care must be taken to provide the appropriate amino acids as well as overall protein content. For example, hogs fed large amounts of corn must have adequate lysine made available in the feed. In most animal diets, energy requirements are met by starches in cereal grains. Energy requirements may also be met by addition of fat to the feed. Animal feeds containing oxidatively transformed carotenoid, a component thereof, or fractionated oxidatively transformed carotenoid may also be formulated for dogs, cats, poultry, fish, and cattle, among others.

Other ingredients may be added to the animal feed as needed to promote the health and growth of the animal. The ingredients include, without limitation, sugars, complex carbohydrates, amino acids (e.g., arginine, histidine, isoleucine, leucine, lysine, methionine, phenylalanine, threonine, tryptophan, valine, tyrosine, alanine, aspartic acid, sodium glutamate, glycine, proline, serine, and cysteine, among others), vitamins (e.g., thiamine, riboflavin, pyridoxine, niacin, niacinamide, inositol, choline chloride, calcium pantothenate, biotin, folic acid, ascorbic acid, and vitamins A, B, K, D, E, among others), minerals, protein (e.g., meat meal, fish meal, liquid or powdered egg, fish solubles, whey protein concentrate), oils (e.g., soybean oil), cornstarch, calcium, inorganic phosphate, copper sulfate, and sodium chloride. Any medicament ingredients known in the art may also be added to the animal feed, including, without limitation, antibiotics and hormones. For vitamin, mineral and antibiotic supplementation of animal feeds see Church, Livestock Feeds and Feeding, O&B Books, Inc., Corvallis Oreg. (1984).

Any animal feed blend known in the art can be used in accordance with the present invention, including, without limitation, forages, such as orchard grass, timothy, tall fescue, ryegrass, alfalfa, sainfoin, clovers and vetches, grain feeds, such as corn, wheat, barley sorghum, triticale, rye, canola, and soya beans, crop residues, cereal grains, legume by-products, and other agricultural by-products. In situations where the resulting feed is to be processed or preserved, the feed may be treated with oxidatively transformed carotenoid, a component thereof, or fractionated oxidatively transformed carotenoid before processing or preservation. Desirably, the animal feed of the invention includes rapeseed meal, cottonseed meal, soybean meal, or cornmeal.

Processing may include drying, ensiling, chopping, pelleting, cubing, baling, rolling, tempering, grinding, cracking, popping, extruding, micronizing, roasting, flaking, cooking, and/or exploding. For example, pelleted feed is created by first mixing feed components and then compacting and extruding the feed components through a die with heat and pressure. Animal feeds of the invention can be pelleted as described in, for example, MacBain, Pelleting Animal Feed, American Feed Manufacturers Association, Arlington, Va. (1974), incorporated herein by reference.

Baked Goods and Beverages

Foodstuffs of the invention can be in the form of a health bar, preferably supplied in foil or other types of wrappers, as is commonly seen in most food markets, convenience stores and health food stores. Typically, such health bars are commonly made by a machine extrusion process that extrudes the mixed ingredients into the desired size and shape bar, which is then conveyed to automatic wrapping machinery. Health bars may be baked, rather than extruded.

The foodstuff may also be extruded, baked, rolled, pressed, cut or otherwise formed into bars or baked goods, such as cookies, brownies, cakes or muffins. In the manufacturing process for bars that are extruded, ingredients such as glycerine, lecithin, vegetable and other oils (such as sunflower oil) are used in part to help bind ingredients together so as to help form a uniformly shaped bar in the extrusion machinery. Such known processes can be used to produce the health bars and baked goods of the present invention.

Foodstuffs of the invention can be in the form of a ready-to-drink beverage, requiring no addition of water and/or mixing with water or other liquids, or a powder or a liquid concentrate that is mixed with water, fruit juice, fruit and/or other flavored drinks, and/or fruit drink concentrates to make, for example, a flavored beverage, or with milk to make a drink having a character similar to that of a milk-shake.

### Dietary Supplements

Alternatively, oxidatively transformed carotenoid, a component thereof, or fractionated oxidatively transformed carotenoid may be administered to a subject as part of a dietary supplement, such as a vitamin supplement, mineral supplement, and/or herbal supplement.

Nutritional additives such as vitamins, vitamin components, and essential nutrients can be used for their known nutritional value as additional ingredients. Thus a vitaminic additive can include any one of, or mixtures of: vitamin A, vitamin C, vitamin D, vitamin E, vitamin K, thiamin, riboflavin, niacin, vitamin B6, folic acid, vitamin B12, biotin, and pantothenic acid, among other vitamins known in the art.

Minerals and mineral components can be used for their nutritional value as additional ingredients. Thus, a mineral additive can include any one of, or mixtures of, the following minerals or nutritionally acceptable elements thereof: calcium, copper, iron, phosphorus, iodine, magnesium, zinc, selenium, copper, manganese, chromium, molybdenum, chloride, potassium, boron, nickel, silicon tin, and vanadium, among other nutritionally important minerals known in the art.

Maintaining adequate levels of vitamins and minerals is essential to health. Many disorders due to vitamin and mineral deficiencies are well known in the art. For example, cognitive decline is a well known problem in the elderly in which diet plays a possible role. Vitamin deficiencies, especially vitamin B6, B12 and folates, and antioxidant deficiencies (vitamins E and C) could also influence the memory capabilities and have an effect on cognitive decline (see Solfrizzi V., et al. The role of diet in cognitive decline. J. Neural Transm. 110:95 (2003)). Minerals are well known to play important roles in the maintenance of health and well-being. Selenium, for example, is a component of glutathione peroxidase, an important natural antioxidant enzyme. As another example of the importance of minerals, insufficient intake of zinc, copper, chromium, and magnesium may affect one's likelihood of developing arteriosclerosis.

Nutritional additives, such as herbs and extracts, can be used in the methods and compositions of the invention. Various processed (e.g., extracts) or unprocessed forms of the following herbs are contemplated as choices for additional nutritional ingredients in the present invention: ginseng, tea (e.g., white tea, green tea, black tea), guarana, gingko, echinacea, cinnamon, chamomile, kola nut, yerba mate, kava kava, yohimbe, elderberry, grape seed, turmeric (curcumin), milk thistle (e.g., silymarin), schisandra, panax quinquefolium, reishi, damiana, chocolate, carob, and other herbs known in the art. These herbs have been used in a variety of formulas for functional energy drinks and health drinks. Chamomile is a well-known folk remedy for insomnia and anxiety. It contains apigenin, which accounts for its anti-anxiety and sedative effects, and works in an analogous way to diazepam. Chocolate has long been known for its ability to improve mood and cognitive function. Cinnamon is known as a digestion aid that can relieve upset stomach, gas, and diarrhea. Elderberry has been shown to be active against influenza, and has long been considered a useful treatment with antiviral activity against colds, herpes, and other virus-related illnesses. Gingko biloba and its extracts have long been studied and used for the prevention and treatment of neurodegenerative pathologies. It also appears to improve mood and cognitive function in some individuals. Ginseng, in its various varieties (e.g., Asian, American, Siberian), is well known as a general health tonic that can increase physical stamina and mental alertness, counter stress, and relieve nervousness and restlessness. Grape seed extracts have been shown to have cardioprotective actions. Furthermore, animal experiments suggest that grape seed extracts can protect against ischemic neuronal damage and, thus, may have neuroprotective properties. Guarana is a common ingredient in many energy drinks and may also be used in the present invention, as can kola nuts and yerba mate. Reishi is a mushroom that has been reported to ease tension, improve memory, and sharpen concentration and focus. In an animal model, chemical constituents of schisandra have been shown to enhance cognitive function.

Any of the vitamins, minerals, herbs, and herbal extracts described herein can be used in the methods and compositions of the invention.

The following examples are put forth so as to provide those of ordinary skill in the art with a complete disclosure and description of how the methods and compositions claimed herein are performed, made, and evaluated, and are intended to be purely exemplary of the invention and are not intended to limit the scope of what the inventors regard as their invention.

### Example 1

### Effect of Oxidatively Transformed Carotenoid on Growth and Feed Conversion in Pigs

Two groups of 48 weaned pigs, ages 18-21 days old, were used to analyze the effects of oxidatively transformed carotenoid as a food additive on growth and feed conversion.

The first 48 pigs were randomly distributed into 16 pens (3 pigs per pen) divided equally between two temperature-controlled rooms. All 24 pigs in one room were injected with an attenuated vaccine against Porcine Respiratory and Reproductive Syndrome (Vaccinated room) and the other 24 pigs were injected a placebo of saline solution (Control room).

Two pens per room were randomly assigned to one of four diets consisting of oxidatively transformed beta-carotene (OxBC) admixed with commercial swine feed.

OxBC was prepared as follows. A suspension of beta-carotene in ethyl acetate at room temperature was saturated with oxygen by bubbling the gas through it while stirring the mixture. After 8 days, when 6 to 8 molar equivalents of oxygen had been consumed, the solvent was evaporated to give a yellow residue of OxBC.

OxBC was mixed with 3 to 10 equivalents by weight of corn starch and ground in a mortar until a homogenous product (by visual inspection) was obtained. The resulting freely flowing powder was further diluted by simple mixing with corn starch and subsequently mixed with a powdered commercial swine feed, the components were milled together, and the mixture pressed into pellets.

The four diets used in the study, diets A-D below, contained OxBC at levels of 0, 10, 30, and 100 mg/kg of swinefeed.

| | |
|---|---|
| Diet A (Control): | Commercial diet with no OxBC |
| Diet B: | Commercial diet with 0.001% (w/w) OxBC |
| Diet C: | Commercial diet with 0.003% (w/w) OxBC |
| Diet D: | Commercial diet with 0.010% (w/w) OxBC |

The pigs had Ad-libitum access to feed and water during the 4-week trial. After a 4 day acclimatization, pigs were individually weighed and placed on the experimental diets for four weeks. Piglets were weighed every 7 days following placement on the diets. All feed given to the pigs was weighed daily, and once per week the feeders were emptied and the feed inventory was weighed.

A sequential replicate of this study was performed. The data were analyzed using a mixed model linear regression with pen as a random effect and start weight as a covariate using software developed by Stata corp.

The growth rate of the pigs was calculated by subtracting the start weight of the pigs from the final weight and dividing by the number of days on the study. These data are summarized in Table 1.

TABLE 1

| | Average Daily Gain |
|---|---|
| OxBC level | (kg ± SE) |
| 0% (w/w) Diet A (control) | 0.535 ± 0.019 |
| 0.001% (w/w) Diet B | 0.578 ± 0.019 |
| 0.003% (w/w) Diet C | 0.540 ± 0.020 |
| 0.010% (w/w) Diet D | 0.507 ± 0.019 |

There was an improvement in growth rate associated with feeding the OxBC product for four weeks after weaning. The effect was statistically significant at 0.001% (w/w) OxBC, where the pigs grew approximately 8% faster than the untreated controls.

The feed conversion was calculated as the weight of the feed consumed in a pen (3 pigs) divided by the weight gained by all three pigs during the study period. These data are summarized in Table 2.

TABLE 2

| | Feed Conversion |
|---|---|
| OxBC level | (kg feed/kg pork ± SE) |
| 0% (w/w) Diet A (control) | 1.65 ± 0.035 |
| 0.001% (w/w) Diet B | 1.51 ± 0.035 |
| 0.003% (w/w) Diet C | 1.63 ± 0.035 |
| 0.010% (w/w) Diet D | 1.56 ± 0.035 |

The feed conversion efficiency of pigs fed for 4 weeks after weaning was increased by the addition of OxBC to the diet. The effect was most pronounced at 0.001% (w/w) OxBC, where the pigs ate approximately 8.5% less feed to gain the same weight.

### Example 2

### Effects of Oxidatively Transformed Carotenoid on Growth Performance in Broiler Chickens

A total of 1600 Ross×Ross 308 cockerel chicks were assigned to treatments at arrival. There were 8 blocks in the study, each comprised of 4 pens. Pens within block were randomly and equally assigned to the treatments (A, B, C, D). There were 50 birds per pen and each pen within a block contained birds of similar initial bodyweight. A randomized complete block design was used to study the effects of the following four treatments in a randomized complete block design:

| | |
|---|---|
| Diet A (Control): | Commercial diet with no OxBC |
| Diet B: | Commercial diet with 0.0005% (w/w) OxBC |
| Diet C: | Commercial diet with 0.001% (w/w) OxBC |
| Diet D: | Commercial diet with 0.003% (w/w) OxBC |

Treatment diets were introduced on Day 0 and were fed continuously until study termination on Day 38. Water was provided ad libitum to birds throughout the trial.

In order to manufacture final feeds, the 20% OxBC cornstarch premix (prepared as described in example 1) was diluted with corn starch to produce a 2% (w/w) OxBC premix. The required amount of active ingredient was delivered by varying the amount of 2% OxBC premix per tonne complete feed.

Pen live weights were recorded on Day 0, 18, 31 and 38 days of age. Pen feed consumption were recorded for periods between days 0-18, 18-31, and 31-38 days of age.

The live weight of birds fed OxBC were significantly higher at day 18 (P=0.010), day 31 (P<0.0001), and at the termination of the trial on day 38 (P=0.022) (see Table 3). No significant differences (P>0.05) were noted between birds fed 5, 10, or 30 ppm OxBC. Birds were 3.7%, 3.0%, and 4.3% heavier after 38 days of feeding 5, 10, and 30 ppm OxBC, respectively, relative to birds fed the control diet.

Feed conversion ratios (FCR) were not significantly (P=0.572) affected over the starter feed period (day 0 to 18). While the FCR of birds fed 10 and 30 ppm were numerically lower than controls, the relative difference was less than 1% (see Table 3). Feed conversion ratios tended (P=0.053) to be significantly improved in birds fed 5 ppm OxBC over the grower period (days 18-31), but not in those fed 10 or 30 ppm OxBC relative to controls. The relative improvement in feed conversion in birds fed 5 ppm OxBC was 3.4%. In contrast, the FCRs were not significantly different among treatments in the finisher period (day 31 to 38; P=0.803), nor over the entire duration of the trial (day 0 to 38; P=0.242). FCRs were similar among all treatments over the entire study despite birds fed OxBC being significantly heavier at the termination of the trial relative to those fed the control diet.

TABLE 3

| | Mean body weight (kg) | | | | Feed conversion ratio (kg/kg gain) | | | |
|---|---|---|---|---|---|---|---|---|
| | Day 0 | Day 18 | Day 31 | Day 38 | D 0-18 | D 18-31 | D 31-38 | D 0-38 |
| 0 ppm control | 0.040 | 0.544 | 1.468 | 2.100 | 1.483 | 1.766 | 2.187 | 1.815 |
| 5 ppm OxBC | 0.040 | 0.575 | 1.553 | 2.178 | 1.484 | 1.706 | 2.224 | 1.792 |
| 10 ppm OxBC | 0.040 | 0.575 | 1.544 | 2.165 | 1.469 | 1.776 | 2.218 | 1.819 |
| 30 ppm OxBC | 0.040 | 0.580 | 1.560 | 2.191 | 1.470 | 1.744 | 2.230 | 1.809 |
| P value | 0.999 | 0.010 | 0.000 | 0.022 | 0.572 | 0.053 | 0.803 | 0.242 |
| Pooled SEM | 0.000 | 0.008 | 0.012 | 0.020 | 0.010 | 0.018 | 0.033 | 0.010 |

The average daily feed intake of birds was significantly improved (P=0.001) over the starter period in birds fed OxBC, with a mean improvement of 5.8% relative to birds fed the control diet (see Table 4). No differences were noted between birds fed 5, 10 or 30 ppm OxBC. Similarly, the average daily feed intake of birds was significantly improved (P=0.016) in birds fed 10, and 30 ppm OxBC over the grower period (days 18 to 31), but not in birds fed 5 ppm OxBC (see Table 4). This is despite a numerically higher feed intake in birds fed 5 ppm OxBC over this time period. No significant differences (P=0.486) were noted in mean feed intakes among treatments in the finisher phase (day 31-38), although they were numerically higher in birds fed OxBC. Pooling the data over the entire production cycle revealed a tendency (P=0.062) toward higher total average daily feed intake in birds fed 10 and 30 ppm OxBC, but not in those fed 5 ppm OxBC.

The average daily gain of birds was significantly (P=0.012) higher in birds fed 5, 10 and 30 ppm OxBC relative to control birds fed starter diets (days 0 to 18), as well as in the grower phase (P<0.0001; days 18 to 31), but not in the finisher phase (P=0.936; days 31 to 38) (see Table 4). Over the entire trial (days 0 to 38), birds fed 5, 10 or 30 ppm OxBC had significantly (P=0.008) higher average daily gains (4.3%, 4.1%, and 5,6%, respectively) relative to birds fed the unsupplemented control diet.

TABLE 4

| | Average Daily Feed Intake (g/day) | | | | Average Daily Gain (g/day) | | | |
|---|---|---|---|---|---|---|---|---|
| | D 0-18 | D 18-31 | D 31-38 | D 0-38 | D 0-18 | D 18-31 | D 31-38 | D 0-38 |
| 0 ppm control | 41.3 | 125.0 | 195.0 | 96.9 | 27.9 | 70.9 | 89.4 | 53.1 |
| 5 ppm OxBC | 43.6 | 128.1 | 197.9 | 99.5 | 29.4 | 75.0 | 89.0 | 55.4 |
| 10 ppm OxBC | 43.4 | 132.0 | 197.4 | 100.5 | 29.6 | 74.4 | 89.0 | 55.3 |
| 30 ppm OxBC | 44.1 | 130.7 | 201.6 | 101.3 | 29.9 | 75.1 | 90.6 | 56.1 |
| P value | 0.001 | 0.016 | 0.486 | 0.062 | 0.012 | 0.000 | 0.936 | 0.008 |
| Pooled SEM | 0.4 | 1.5 | 3.0 | 1.1 | 0.4 | 0.7 | 2.1 | 0.6 |

Dietary supplementation with OxBC significantly improved the mean final body weights of birds by 3.7% (5 ppm), 3.0% (10 ppm), and 4.3% (30 ppm) after 38 days of growth under normal rearing conditions. Average feed intakes tended to be improved, while average daily gains were significantly improved with OxBC dietary supplementation.

### Example 3

### Dose Optimization Study in Pigs

A total of 240 pigs were assigned to treatment at weaning. Eight blocks were used in the study, each comprised of five pens. Pens within each block were randomly and equally assigned to one of the diets (A, B, C, D, or E). There were six pigs per pen.

The five diets used in the pig dose optimization study, diets A-E below, contained OxBC at levels of 0, 0, 1, 2, and 5 mg/kg of swinefeed.

| | |
|---|---|
| Diet A (Control): | Commercial diet with no OxBC |
| Diet B (Control): | Commercial diet with no OxBC + medicated |
| Diet C: | Commercial diet with 0.0001% (w/w) OxBC |
| Diet D: | Commercial diet with 0.0002% (w/w) OxBC |
| Diet E: | Commercial diet with 0.0005% (w/w) OxBC |

The pigs had Ad-libitum access to feed and water during the 35 day trial. No in-feed or water-administered medication was used in the trial, except for treatment B, which contained antibiotics.

The live weight of pigs fed OxBC were not significantly (P>0.05) different to the unmedicated control (0 ppm control) at any sampling point in the trial (see Table 5). These results suggest that the highest level of OxBC used in the dose optimization study, 5 ppm OxBC, is not sufficient to elicit a significant improvement in growth of pigs under commercial production conditions.

TABLE 5

| | Mean body weight (kg) | | | | | |
|---|---|---|---|---|---|---|
| | Day 0 | Day 7 | Day 14 | Day 21 | Day 28 | Day 35 |
| 0 ppm control | 7.25 | 8.08 | 9.51^{b} | 12.18^{ab} | 15.26 | 18.32 |
| 0 ppm Med (B) | 7.28 | 8.17 | 10.08^{a} | 12.88^{a} | 15.65 | 18.33 |
| 1 ppm OxBC | 7.22 | 7.81 | 9.26^{b} | 11.51^{b} | 13.99 | 17.05 |
| 2 ppm OxBC | 7.22 | 7.96 | 9.44^{b} | 12.16^{ab} | 15.23 | 18.17 |
| 5 ppm OxBC | 7.27 | 8.06 | 9.64^{ab} | 12.23^{ab} | 15.25 | 18.39 |
| P value | 0.504 | 0.305 | 0.036 | 0.041 | 0.059 | 0.323 |
| Pooled SEM | 0.04 | 0.15 | 0.18 | 0.29 | 0.39 | 0.51 |
| P-value Block | 0.000 | 0.000 | 0.000 | 0.000 | 0.000 | 0.000 |

¹Values within a column bearing a common letter are not significantly different (P > 0.05).

No differences (P>0.05) in overall feed intake were observed. Feed conversion ratios were not significantly (P=0.528) affected over the initial feed period (day 0 to 7). Feed conversion ratios of pigs fed the medicated control (0 ppm OxBC) were significantly (P<0.05) lower than pigs fed the unmedicated control, 2 ppm OxBC and 5 pp OxBC between day 7 and 14, but not different to those fed 1 ppm OxBC (see Table 6). In contrast, between day 21 and 28, pigs fed 1 ppm OxBC had a significantly higher (P<0.05) feed conversion ratio to all other treatments. Overall, the feed conversion ratios tended (P=0.075) to be significantly different between treatments, with pigs fed the medicated control showing the numerically lowest feed conversion.

For the overall growth period, pigs fed 2 ppm OxBC had a numerically (3.6%) lower feed conversion, and pigs fed 5 ppm OxBC a 1.1% lower feed conversion relative to pigs fed the unmedicated 0 ppm OxBC control (see Table 6).

TABLE 6

| | Feed Conversion Ratio | | | | | |
|---|---|---|---|---|---|---|
| | Day 0-7 | Day 7-14 | Day 14-21 | Day 21-28 | Day 28-35 | Day 0-35 |
| 0 ppm control | 0.878 | 1.509^{a} | 1.459 | 1.582^{a} | 1.959 | 1.595 |
| 0 ppm Med (B) | 1.480 | 1.298^{b} | 1.362 | 1.649^{a} | 1.859 | 1.506 |
| 1 ppm OxBC | 1.381 | 1.445^{ab} | 1.446 | 1.925^{b} | 1.770 | 1.676 |
| 2 ppm OxBC | 1.863 | 1.455^{a} | 1.350 | 1.496^{a} | 1.892 | 1.537 |
| 5 ppm OxBC | 2.943 | 1.514^{a} | 1.424 | 1.572^{a} | 1.826 | 1.577 |
| P value | 0.528 | 0.051 | 0.597 | 0.025 | 0.823 | 0.075 |
| Pooled SEM | 0.859 | 0.053 | 0.059 | 0.091 | 0.115 | 0.042 |
| P-value Block | 0.963 | 0.032 | 0.344 | 0.147 | 0.694 | 0.022 |

¹Values within a column bearing a common letter are not significantly different (P > 0.05).

### Example 4

### Dose Optimization Study in Chickens

A total of 2500 chicks were assigned to treatment at arrival. Five blocks were used in the study, each comprised of ten pens. Pens within each block were randomly and equally assigned to one of the diets (A, B, C, D, or E).

The five diets used in the chicken dose optimization study, diets A-E below, contained OxBC at levels of 0, 0, 1, 2, and 5 mg/kg of feed (starter feed on days 0-18, grower feed on days 18-30, and finisher feed on days 30-38). Pen live weights were recorded on Day 0, 18, 31, and 39 days of age.

| | |
|---|---|
| Diet A (Control): | Commercial diet with no OxBC |
| Diet B (Control): | Commercial diet with no OxBC + medicated |
| Diet C: | Commercial diet with 0.0001% (w/w) OxBC |
| Diet D: | Commercial diet with 0.0002% (w/w) OxBC |
| Diet E: | Commercial diet with 0.0005% (w/w) OxBC |

Water was provided ad libitum to birds throughout the 39 day trial. No in-feed medication was used in the trial, except for treatment B, which contained an antibiotic.

The live weight of birds fed the medicated control diet were significantly higher compared to birds fed the non-medicated control, 1 ppm, or 2 ppm OxBC at day 18 (P=0.022)(see Table 7). Birds fed 2 and 5 ppm OxBC had significantly higher live weights at the termination of the trial on day 39 (P=0.002). Broilers fed 1 ppm OxBC, while not significantly different from those fed the 0 ppm control diets (medicated or non-medicated), were numerically higher.

No significant gender-treatment interaction was noted on body weights (P>0.100), suggesting that dietary OxBC affected both genders equally.

Birds were 3.2%, 4.5%, and 3.6% heavier after 39 days of feeding 1, 2, and 5 ppm OxBC, respectively, relative to birds fed either the non-medicated or medicated control diets.

TABLE 7

| | Mean body weight (kg) | | | |
|---|---|---|---|---|
| | Day 0 | Day 18 | Day 31 | Day 39 |
| 0 ppm control | 0.044 | 0.605^{a} | 1.739 | 2.184^{c} |
| 0 ppm + Med control | 0.044 | 0.588^{b} | 1.724 | 2.182^{c} |
| 1 ppm OxBC | 0.044 | 0.604^{a} | 1.710 | 2.216^{bc} |
| 2 ppm OxBC | 0.044 | 0.605^{a} | 1.751 | 2.282^{a} |
| 5 ppm OxBC | 0.044 | 0.594^{ab} | 1.727 | 2.262^{ab} |
| P value | 0.993 | 0.022 | 0.157 | 0.002 |
| Pooled SEM | 0.000 | 0.004 | 0.012 | 0.020 |
| Block - P-value | 0.071 | 0.028 | 0.138 | 0.045 |
| Pullets | 0.044 | 0.583 | 1.847 | 2.389 |
| Cockerels | 0.044 | 0.616 | 1.613 | 2.061 |
| P value | 0.146 | 0.000 | 0.000 | 0.000 |
| Pooled SEM | 0.000 | 0.003 | 0.007 | 0.013 |
| Gender * Trt | 0.829 | 0.152 | 0.662 | 0.685 |
| P-value | | | | |

¹Values within a column bearing a common letter are not significantly different (P > 0.05).

Feed conversion ratios were not significantly (P=0.129) affected over the starter feed period (day 0 to 18). Feed conversion ratios were significantly (P=0.040) poorer in birds fed 1 ppm OxBC over the grower period (days 18-31), but not in those fed 2 or 5 ppm OxBC relative to those fed the non-medicated control diet (see Table 8), while birds fed 2 ppm OxBC had a lower FCR compared to birds fed the medicated control. In contrast, FCRs were significantly lower in birds fed any level of OxBC in the finisher period (day 31 to 39; P=0.001), compared to birds fed either control diet. Birds fed dietary OxBC had lower FCRs compared to the non-medicated control diet over the entire duration of the trial (day 0 to 39; P=0.018), while only birds fed 5 ppm OxBC had a significantly lower FCR compared to those fed the medicated control diet.

TABLE 8

| | Feed conversion ratio (kg/kg gain) | | | |
|---|---|---|---|---|
| | D 0-18 | D 18-31 | D 31-39 | D 0-39 |
| 0 ppm control | 1.469 | 1.665^{bc} | 2.872^{a} | 1.851^{a} |
| 0 ppm + Med control | 1.484 | 1.650^{c} | 2.781^{a} | 1.831^{ab} |
| 1 ppm OxBC | 1.462 | 1.701^{a} | 2.429^{b} | 1.806^{bc} |
| 2 ppm OxBC | 1.466 | 1.684^{ab} | 2.389^{b} | 1.789^{bc} |
| 5 ppm OxBC | 1.520 | 1.670^{abc} | 2.328^{b} | 1.788^{c} |
| P value | 0.129 | 0.040 | 0.001 | 0.018 |
| Pooled SEM | 0.017 | 0.012 | 0.103 | 0.015 |
| Block - P-value | 0.517 | 0.106 | 0.136 | 0.179 |
| Pullets | 1.475 | 1.647 | 2.536 | 1.795 |
| Cockerels | 1.486 | 1.701 | 2.584 | 1.831 |
| P value | 0.481 | 0.000 | 0.607 | 0.009 |
| Pooled SEM | 0.011 | 0.007 | 0.065 | 0.009 |
| Gender * Trt | 0.522 | 0.219 | 0.769 | 0.453 |
| P-value | | | | |

¹Values within a column bearing a common letter are not significantly different (P > 0.05).

The average daily feed intake of birds was significantly improved (P=0.001) over the starter period (days 0-18) in birds fed 2 ppm OxBC, with a mean improvement of 2.1% relative to birds fed the control diets of those fed 1 or 5 ppm OxBC (see Table 9). No differences were noted in feed intake between treatments throughout the grower period (days 18-31; P=0.278). No significant differences (P=0.486) were noted in mean feed intakes among treatments in the finisher phase (day 31-39), or over the entire production cycle (days 0-39; P=0.328).

TABLE 9

| | Average Daily Feed Intake (g/day) | | | |
|---|---|---|---|---|
| | Day 0 | Day 18 | Day 31 | Day 39 |
| 0 ppm control | 45.5 | 134.2^{b} | 208.5 | 103.2 |
| 0 ppm + Med control | 44.7 | 133.4^{b} | 204.3 | 102.4 |
| 1 ppm OxBC | 45.3 | 134.2^{b} | 203.4 | 102.6 |
| 2 ppm OxBC | 45.3 | 137.1^{a} | 206.5 | 104.1 |
| 5 ppm OxBC | 46.2 | 134.3^{b} | 206.3 | 103.6 |
| P value | 0.444 | 0.071 | 0.278 | 0.328 |
| Pooled SEM | 0.6 | 0.9 | 1.7 | 0.6 |
| Block - P-value | 0.435 | 0.108 | 0.238 | 0.124 |
| Pullets | 46.5 | 144.1 | 222.7 | 109.5 |
| Cockerels | 44.2 | 125.2 | 188.9 | 96.8 |
| P value | 0.000 | 0.000 | 0.000 | 0.000 |
| Pooled SEM | 0.3 | 0.6 | 1.1 | 0.4 |
| Gender * Trt | 0.331 | 0.221 | 0.490 | 0.087 |
| P-value | | | | |

The average daily gain of birds was significantly (P=0.049) higher in birds fed 0, 1, and 2 ppm OxBC relative to birds fed the non-medicated control starter diet (days 0 to 18). Birds fed OxBC had significantly higher average daily gains compared to those fed either control diet in the finisher phase (P=0.001; days 31 to 39)(see Table 10). Over the entire trial (days 0 to 39), birds fed 2, or 5 ppm OxBC had significantly (P=0.004) higher average daily gains (4.1% and 3.8%, respectively) relative to birds fed either control diet, with birds fed 1 ppm OxBC having intermediate gains that were not significantly different from birds fed other treatments.

TABLE 10

| | Average Daily Gain (g/day) | | | |
|---|---|---|---|---|
| | D 0-18 | D 18-31 | D 31-39 | D 0-39 |
| 0 ppm control | 31.1^{a} | 80.7 | 74.1^{b} | 56.0^{b} |
| 0 ppm + Med control | 30.1^{b} | 81.1 | 75.8^{b} | 55.9^{b} |
| 1 ppm OxBC | 30.9^{a} | 79.1 | 84.0^{a} | 57.1^{ab} |
| 2 ppm OxBC | 30.9^{a} | 81.7 | 87.2^{a} | 58.3^{a} |
| 5 ppm OxBC | 30.4^{ab} | 80.6 | 89.0^{a} | 58.1^{a} |
| P value | 0.049 | 0.129 | 0.001 | 0.004 |
| Pooled SEM | 0.0 | 0.7 | 2.8 | 0.5 |
| Block - P-value | 0.122 | 0.095 | 0.076 | 0.061 |
| Pullets | 31.5 | 87.6 | 89.9 | 61.2 |
| Cockerels | 29.8 | 73.6 | 74.1 | 53.0 |
| P value | 0.000 | 0.000 | 0.000 | 0.000 |
| Pooled SEM | 0.0 | 0.4 | 1.8 | 0.3 |
| Gender * Trt | 0.065 | 0.725 | 0.637 | 0.664 |
| P-value | | | | |

Taken together, the present data suggests that dietary supplementation with 2 or 5 ppm OxBC over the finisher period significantly enhances growth.

### Other Embodiments

All publications and patent applications, and patents mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims.

What we claim is:

1. A foodstuff comprising an additive selected from oxidatively transformed carotenoid and fractionated oxidatively transformed carotenoid.

2. The foodstuff of claim 1, wherein said additive is fractionated oxidatively transformed carotenoid.

3. The foodstuff of claim 2, wherein said fractionated oxidatively transformed carotenoid comprises the polymeric component of oxidatively transformed carotenoid.

4. The foodstuff of claim 2, wherein said fractionated oxidatively transformed carotenoid comprises a mixture of compounds, each of said compounds having a molecular weight of less than 700 Da.

5. The foodstuff of claim 2, wherein said fractionated oxidatively transformed carotenoid comprises a mixture of compounds, each of said compounds having a molecular weight of less than 300 Da.

6-18. (canceled)

19. A kit, comprising:
(i) a composition comprising a food additive selected from oxidatively transformed carotenoid and fractionated oxidatively transformed carotenoid; and
(ii) instructions for administering said additive to an animal.

20. The kit of claim 19, wherein said composition comprises a bulking agent and from 0.5% to 50% (w/w) of said food additive.

21. The kit of claim 19, further comprising instruction for mixing said composition with an animal feed.

22. The kit of claim 19, wherein said additive is fractionated oxidatively transformed carotenoid.

23. The kit of claim 19, wherein said fractionated oxidatively transformed carotenoid comprises the polymeric component of oxidatively transformed carotenoid.

24. The kit of claim 19, wherein said fractionated oxidatively transformed carotenoid comprises a mixture of compounds, each of said compounds having a molecular weight of less than 700 Da.

25. The kit of claim 19, wherein said fractionated oxidatively transformed carotenoid comprises a mixture of compounds, each of said compounds having a molecular weight of less than 300 Da.

26-27. (canceled)

28. A food supplement comprising:
a) a vitamin selected from vitamin C, vitamin D, vitamin E, vitamin K, folate, vitamin B6, and vitamin B12; and
b) oxidatively transformed carotenoid or a component thereof.

29. The food supplement of claim 24, wherein said supplement is formulated in a unit dosage form containing from about 5% to about 1000% of the RDA of said vitamin and from about 100 μg to 100 mg of said oxidatively transformed carotenoid or a component thereof.

30. A food supplement comprising:
a) a mineral selected from calcium, chromium, copper, fluoride, iodine, iron, magnesium, manganese, molybdenum, phosphorus, potassium, selenium, sodium, and zinc; and
b) oxidatively transformed carotenoid or a component thereof.

31. The food supplement of claim 30, wherein said supplement is formulated in a unit dosage form containing from about 5% to about 500% of the RDA of said mineral and from about 100 μg to 100 mg of said oxidatively transformed carotenoid or a component thereof.

32. A food supplement comprising:
a) an omega fatty acid selected from alpha-linolenic acid, stearidonic acid, eicosatetraenoic acid, eicosapentaenoic acid, docosahexaenoic acid, linoleic acid, gamma-linolenic acid, dihomo-gamma-linolenic acid, and arachidonic acid; and
b) oxidatively transformed carotenoid or a component thereof.

33. The food supplement of claim 32, wherein said supplement is formulated in a unit dosage form containing from about 10 mg to 2 g of said omega fatty acid and from about 100 μg to 100 mg of said oxidatively transformed carotenoid or a component thereof.

34. A food supplement comprising:
a) an amino acid selected from isoleucine, leucine, lysine, methionine, phenylalanine, threonine, tryptophan, and valine; and
b) oxidatively transformed carotenoid or a component thereof.

35. The food supplement of claim 34, wherein said supplement is formulated in a unit dosage form containing from about 5% to 500% of the RDA of said amino acid and from about 100 μg to 100 mg of said oxidatively transformed carotenoid or a component thereof.

36. A food supplement comprising:
a) an herb selected from angelica, astragalus, avena sativa, bayberry bark, billberry, black cohosh, black haw, black walnut, blessed thistle, blue cohosh, blue vervain, buchu, buckthorn, burdock, cascara sagada, casteberry, cayenne, chamomille, chaparral, chaste tree, chickweed, cloves, coltsfoot, comphrey root, cornsilk, cough calm, crampbark, damiana, dandelion, dandelion root, dill seed, dong quai, echinacea, elecampane, essiac, eucalyptus, fennel, fenugreek, gentian, ginger, ginkgo, ginseng, goldenseal, gota kola, guarana, hawthorne berry, hops, horehound, horsetail, hydrangea, hyssop, kelp, kola nut, licorice, lobelia, maca, marshmallow, motherwort, muira puama, mullien, myrrh, nettle, oatstraw, oregon grape root, parsley, passion flower, pau d'arco, peppermint, plantain, pleurisy root, prickley ash bark, red clover, red raspberry, sarsaparilla, saw palmetto, schizandra, scullcap, sheep sorrel, slippery elm, squawvine, St. Johns wort, tumeric, turkey rhubarb, valerian, white willow bark, wild cherry bark, wild yam, yarrow, yellow dock, yohimbi, and extracts thereof; and
b) oxidatively transformed carotenoid or a component thereof.

37. The food supplement of claim 36, wherein said supplement is formulated in a unit dosage form containing from about 1 mg to 250 mg of said herb and from about 100 μg to 100 mg of said oxidatively transformed carotenoid or a component thereof.

38. A food supplement formulated in unit dosage form containing from 100 μg to 100 mg oxidatively transformed carotenoid or a component thereof.

39. The food supplement of claim 38, wherein said unit dosage form is a tablet, pill, capsule, or caplet.

40-46. (canceled)

47. A kit, comprising:
(i) a food supplement of claim 38, and
(ii) instructions for administering said food supplement to an animal.

48-49. (canceled)
